(19) United States
(12) Patent Application Publication
(10) Pub. No.: US 2016/0058752 A1
(43) Pub. Date: Mar. 3, 2016

(54) TOPICAL PERIPHERAL NEURO-AFFECTIVE (TPNA) THERAPY FOR NEUROPATHIC CONDITIONS

(71) Applicant: Ronald Aung-Din, Sarasota, FL (US)

(72) Inventor: Ronald Aung-Din, Sarasota, FL (US)

(73) Assignee: AFGIN PHARMA, LLC, Sarasota, FL (US)

(21) Appl. No.: 14/634,137

(22) Filed: Feb. 27, 2015

Related U.S. Application Data
(60) Provisional application No. 61/935,553, filed on Feb. 4, 2014.

(51) Int. Cl. A61K 31/485; A61K 31/714; A61K 31/519; A61K 45/06; A61K 31/135; A61K 31/381; A61K 9/00; A61K 9/06; A61K 31/4409; A61K 31/675

(52) U.S. Cl. CPC A61K 31/485; A61K 31/4409; A61K 31/714; A61K 31/519; A61K 31/675; A61K 31/135; A61K 31/381; A61K 9/0014; A61K 9/06; A61K 9/0019; A61K 45/06

(57) ABSTRACT

Formulations and methods of treating peripheral neuropathic conditions in humans is disclosed.

This application claims priority to U.S. Provisional Application No. 61/935,553, filed on Feb. 4, 2014, the disclosure of which is hereby incorporated by reference herein.

### FIELD OF THE INVENTION

The invention relates to topical peripheral neuro-affective therapy (“TPNA THERAPY”) for the treatment of neuropathic conditions which affect peripheral nerves.

### BACKGROUND OF THE INVENTION

Topical Regional Neuro-Affective (TRNA, and also known as Nuchal Topical Neuro-Afferent Therapeutics, NT) therapy using “Central Nervous System (CNS)-active” drugs for the treatment of brain electro-chemical dysfunctional states has been previously described. The inventor's previous U.S. Patent Publication No. 20030013753 (filed Jun. 5, 2002) and U.S. Patent Publication No. 20080090894, both of which are hereby incorporated by reference, disclose a unit dose of a topical formulation for treating a migraine or cluster headache comprising: a serotonin agonist incorporated into a pharmaceutically acceptable vehicle for topical administration onto the skin, specifically at the back of neck/nape at the hairline (BONATH) of a human patient. This is in order to capitalize on the relationship that exists at this site with respect to the cutaneous free nerve-endings and the afferent nerves relaying back to the Central Nervous System, not found elsewhere. Preferably, the unit dose providing the serotonin agonist is in a form that is immediately absorbable when said unit dose is applied onto human skin. Preferably, the serotonin agonist comprises from about 0.5 to about 200 mg of sumatriptan, by weight based on the succinate salt, or a therapeutic equivalent dose of another topically absorbable pharmaceutically acceptable serotonin agonist. Preferably, the unit dose provides relief from a migraine or cluster headache within about 2 hours after topical administration to a human patient.

The inventor's previous U.S. Patent Publication No. 20070065463 (filed Jun. 21, 2004) discloses a topical formulation for treating migraines or cluster headaches, muscle sprains, muscle spasms, spasticity, tension headaches, tension related migraines and related conditions associated with muscle tension and pain comprising: a therapeutically effective amount of an active agent(s) incorporated into a pharmaceutically acceptable excipient for topical administration onto the skin of a human patient, the active agent(s) being selected from the group consisting of: i) an ergot alkaloid; ii) a skeletal muscle relaxant; or iii) a combination of an ergot alkaloid and a skeletal muscle relaxant; the active agent(s) being present in an effective concentration such that a unit dose of the topical formulation provides a therapeutic effect within about 2 hours after topical administration to the human patient. In certain preferred embodiments, the topical formulation comprises a skeletal muscle relaxant such as tizanidine. In certain preferred embodiments, the unit dose comprises from about 0.4 mg to 8 mg, preferably from about 0.2 mg to about 4 mg of tizanidine hydrochloride.

In these methods, active drug compounded in an appropriate “dermal penetration-enhancing medium” is applied at the back of the neck, the nape or nuchal region, at the hairline (“BONATH”), capitalizing on the specific relationship of the cutaneous free nerve-endings at this location with the afferent neural systems of the trigeminal, vagal, and sympathetic nerves with the CNS. By affecting afferent neural input (nerve impulses returning from the periphery/body to the brain), CNS efferent neural output (nerve impulses generated by the brain and outgoing to the body), as clinical symptoms, is modulated. Depending on the specific neuroanatomy and neurochemical processes involved in the pathologic or dysfunctional state, clinical symptoms may vary in presentation. With migraine, they are headache, nausea, visual obscurations, and dizziness as the trigeminal nerve and the serotonergic systems are involved.

Alternatively, in Parkinson′ disease (PD), the dopaminergic system within the nigra-striatal pathways is affected. The clinical symptoms in this case are tremor, rigidity, postural instability, and; bradykinesia and bradyphrenia (slowed movements and thought processes), Muscle pain and depression are also common.

The inventor's previously filed International Patent Application WO 2010/005507 discloses a method of treating a disease state or condition in humans with a drug comprising administering a drug selected from the group consisting of anti-epileptic, an anxiolytic, a neuroleptic, an anti-psychotic, an analgesic, an anti-inflammatory, an anti-Parkinson's disease/syndrome drug, a sexual dysfunction drug, a drug for the treatment of dystonia, a drug for the treatment of spastic conditions, a drug for the treatment of benign essential/familial tremor, a drug for the treatment of tremor, a drug for the treatment of chronic encepahalopathies, a drug for the treatment of congenital CNS degeneration conditions/cerebral palsy, a drug for the treatment of cerebellar degeneration syndromes, a drug for the treatment of neuropathic and/or neurogenic pain, a drug for smoking cessation, a drug for appetite suppression, a drug for neurodegenerative conditions, a drug for the treatment of multiple sclerosis, a drug for the treatment of insomnia, a drug for the treatment of fatigue, a drug for the treatment of vertigo, nausea and/or dizziness, a drug for the treatment of writer's cramp and restless leg syndrome, a drug for the treatment of ADD/ADHD, in a therapeutically effective amount to treat the disease state or condition, to the back of the neck at the hairline in close proximity to and under or on the area of skin above the brain stem to provide regional neuro-affective therapy to the patient. Thus, TRNA drug delivery, applied at the back of the neck at the hairline (“BONATH”) may potentially be used for any disease state which has as its basis a dysfunctional electro-chemical state within the brain. Besides migraine and PD, other conditions shown amenable to TRNA therapy include: tension headache, tremor of various etiologies, spastic conditions from brain injury, seizure disorders; and mood disorders such as depression, anxiety, psychotic states, and bi-polar affective disorder. Also included are other brain-derived disorders such as erectile dysfunction, restless legs syndrome, OCD and compulsive behaviors such as gambling, addictions, and hypersexual states. These latter conditions involve dysfunction within the dopamine neurochemical system.

Topical Peripheral Neuro-Affective (TPNA) has been previously described in the inventor's previous U.S. Patent Publication No. 2013/0165468, filed Mar. 12, 2013, hereby incorporated by reference.

### SUMMARY OF THE INVENTION

It is an object of the present invention to provide a method for the treatment of neuropathic conditions.

It is a further object of the present invention to provide a method for the treatment and/or reversal of part or all of the symptoms of neuropathic conditions in mammals, particularly humans.

It is an object of the present invention to provide a novel treatment for peripheral neuropathic conditions which preferably by-passes blood & blood-brain-barrier and provides rapid therapeutic effects, while causing minimized side-effects, and preferably with substantially reduced or substantially no measurable presence in blood, direct effects of the drugs are enhanced over traditional (e.g., oral) therapy, as side-effect, drug-drug interactions and excretion into the environment are substantially reduced or eliminated.

In accordance with the above objects and others, the invention is directed in part to a method of treating neuropathic conditions in humans comprising applying a therapeutically effective amount of a drug selected from the group consisting of a dopamine agonist, a drug which enhances conduction of damaged demyelinated nerves (e.g., a drug which blocks leaking potassium channels in damaged demyelinated nerves to improve conduction), and a combination thereof at the site of the injury, e.g., topically to the site of the injury.

The present invention is further directed in part to a method of treating peripheral neuropathic conditions.

The present invention is further directed in part to a method of treating diabetic neuropathy in humans at the site of the injury, e.g., topically to the site of the injury.

The invention is further directed in part to the invention is directed in part to a method of treating muscle spasm in humans resulting from a peripheral nerve injury comprising applying a therapeutically effective amount of a drug selected from the group consisting of a dopamine agonist, a skeletal muscle relaxant, an opioid agonist, an SNRI, a local anesthetic agent, or an NMDA antagonist; and any combination thereof topically to the site of the injury. In certain preferred embodiments, the injury is neuronal hyperexcitability and/or a neurochemical dysfunction syndrome.

Certain embodiments of the present invention are directed to a method of treating generalized and/or isolated peripheral neuropathy conditions by applying a therapeutically effective amount of a drug selected from the group consisting of a dopamine agonist, a drug which enhances conduction of damaged demyelinated nerves (e.g., a drug which blocks leaking potassium channels in damaged demyelinated nerves to improve conduction), and any combination thereof at two or more sites along the nerve, preferably concurrently. In further aspects of the present invention, it is contemplated that the treatment encompass two or three application sites, depending on the disease state and the place of injury. The drug (same or different drug) is applied as two or three full doses or one-two divided doses at the application sites.

For purposes of the present invention, a “topical formulation” includes, for example, ointments, creams, lotions, pastes, gels, etc., which releases one or more drugs (e.g., dopamine agonists) at a predetermined rate over a defined period of time to a defined site of application.

For purposes of the present invention, an “injectable” formulation includes, for example, an injectable solution, suspension, gel or the like and may be in immediate release form or may provide a controlled or sustained release of the drug at the site of administration.

For purposes of the present invention, an “implantable” formulation includes, for example, a solid, semisolid or liquid drug formulation which can be administered at the site of administration either via injection and/or via surgical implantation. The solid may comprise microspheres, microcapsules, pellets, discs, and the like. The implantable formulations of the invention may provide a controlled or sustained release of the drug at the site of administration.

For purposes of the present invention, a “transdermal therapeutic system” is defined as a drug-containing device (including e.g., patch, disc, etc.) which releases one or more drugs at a predetermined rate over a defined period of time to a defined site of application.

For purposes of the present invention, “transdermal” delivery is the delivery by passage of a drug through the skin and into the bloodstream (“traditional” transdermal delivery) and is termed “transdermal systemic drug delivery (TSD therapy).

For purposes of the present invention, the term “topical transdermal therapy” is synonomous with the more accurately termed topical peripheral neuro-affective therapy (or “TPNA therapy”).

For purposes of the present invention “therapeutically effective” or “effective” amount is meant to be a nontoxic but sufficient amount of a compound to provide the desired therapeutic effect, e.g., allevation or partial or full reversal of the symptoms of a neurological condition.

For purposes of the present invention, the term “delivers” when used with respect to the topical formulation or transdermal therapeutic system means that the formulation or system provides a mean relative release rate or flux of the drug out of the formulation or system and through the skin of the patient.

By “predetermined area of skin” is intended a defined area of intact unbroken living skin. In certain embodiments of the present invention, the predetermined area will be in the range of about 1 cm2 to about 100 cm2, preferably in the range of about 10 cm2 to about 100 cm2, more preferably in the range of about 20 cm2 to about 60 cm2. However, it will be appreciated by those skilled in the art of topical delivery that the area of skin through which drug is administered may vary significantly, depending on the formulation, dose, the application of the formulation, and the like.

“Penetration enhancement” or “permeation enhancement” for purposes of the present invention relates to an increase in the permeability of skin to a pharmacologically active agent, i.e., so as to increase the rate at which the drug permeates through the skin and enters the bloodstream. The enhanced permeation effected through the use of such enhancers can be observed by measuring the rate of diffusion of drug through animal or human skin using a diffusion cell apparatus. An “effective” amount of a permeation enhancer as used herein, for example, means an amount that will provide the desired increase in skin permeability and, correspondingly, the desired depth of penetration, rate of administration, and amount of drug to be delivered.

For purposes of the present invention, the “brainstem afferent stimulation therapy region” is defined as the skin region of the head and/or at the frontotemporal region and/or upper posterior cervical area. In certain preferred embodiments, the treatment area is the post cervical area in close proximity to the brain stem. Preferably this area is a relatively hairless area of the patient's head and/or neck.

For purposes of the present invention, the phrase “to the site of the injury” means that the pharmaceutical formulation including the drug is applied topically or administered via injection directly at the injured structure or in proximity thereto.

For purposes of the present invention, the drug may be in the form of the base, or may be provided as a pharmaceutically acceptable salt (inorganic or organic) or complex. It may be in an optically pure form or a mixture of stereoisomers.

### BRIEF DESCRIPTION OF THE DRAWINGS

FIG. 1 is a picture of a front view of the feet of a patient suffering from Charcot-Marie-Tooth peripheral neuropathy ten minutes after the patient was treated with topical 3 aminopyridine compounded cream to the left ankle and foot.

FIG. 2 is a picture of a front view of the feet of a patient suffering from Charcot-Marie-Tooth peripheral neuropathy prior to and after the patient was treated with topical 4 amino pyridine compounded cream to the left ankle and foot.

FIG. 3 is a picture of a front view of the feet of a patient suffering from diabetic peripheral neuropathy ten minutes after the patient was treated with topical 4 amino pyridine compounded cream to the right ankle and foot.

### DETAILED DESCRIPTION

The present invention is directed in part to the fact that TPNA may be applied to areas of the body affected or injured as a result of a neurological condition.

The term “neuropathic condition” or “neuropathy” as used in the present invention is meant to encompass (but not be limited to) diabetic peripheral neuropathy (DPN); chemotherapy related neuropathies; hereditary neuropathies (Charcot-Marie-Tooth, etc.); metabolic neuropathies such as related to B12 deficiency; thyroid disease; compressive or post-traumatic neuropathies such as carpal tunnel, tarsal tunnel, and cubital tunnel syndromes; as well as compression at fibula head (peroneal neuropathy) and the inguinal ligament of the lateral femoral cutaneous nerve (meralgia paresthetica).

The present invention in certain preferred embodiments is directed to peripheral neuropathic conditions. Such conditions include but are not limited to diabetic peripheral neurophathy (“DPN”) and other peripheral neuropathic conditions.

DPN is Nerve damage and dysfunction secondary to diabetes mellitus type 1 or 2 patients. A consensus definition of DPN is “the presence of symptoms and/or signs of peripheral nerve dysfunction in people with diabetes after exclusion of other causes.” DPN is a leading cause of neuropathic pain and a very common complication of diabetes. DPN may occur in 50-90% of patients depending on the criteria used for diagnosis. While there are a wide range of treatments available for neuropathic pain, the prescribing patterns suggest that there is no one treatment that addresses all of the factors and many patients remain inadequately treated in several aspects of the disease, despite a spectrum of drugs available with different modes of action.

Signs and symptoms of DPN include, but are not limited to the following: distal symmetrical sensorimotor polyneuropathy is the most common form of DPN. Signs and symptoms may progress from distal to proximal over time. Signs include diminished vibratory perception, decreased knee and ankle reflexes, reduced protective sensation, such as pressure, hot and cold, pain; diminished ability to sense position of toes and feet. Symptoms include numbness, loss of feeling, prickling, tingling, aching pain, burning pain, lancinating pain, and unusual sensitivity or tenderness when feet are touched (allodynia). Positive symptoms of DPN include spontaneous pain, dysesthesias, paresthesias. Negative symptoms include loss/impairment of sensory quality, numbness, dry skin, erectile dysfunction, incontinence, gait instability and fall risk. Positive and negative symptoms have an impact on functioning, activities of daily living (ADL) and quality of life (QOL). DPN patients may exhibit psychosocial morbidity (depression, anxiety, anger and loss of self-esteem), societal consequences (social isolation, strained relationships with family and friends, and effects upon intimacy/sexual activity). Examination of tissues from patients with diabetes reveals capillary damage, including occlusion in the vasa nervorum. Reduced blood supply to the neural tissue results in impairments in nerve signaling that affect both sensory and motor function. There is also endothial dysfunction in DPN.

Sensorimotor “peripheral” neurophathy is the most common type of diabetic neuropathy, affecting 30-50% of the entire diabetic population, and is the most commonly involved symptom in diabetic foot problems. The symptoms are development progressive, initially involving more distal parts. The main symptoms are numbness of the legs and feet, muscular cramps, pins and needles, shooting, deep aching and burning pain. Nocturnal exacerbation is characteristic. The symptoms may be absent or present either in the early or late stages.

Small fiber neuropathy may be diagnosed with a skin punch biopsy. Small Fiber Neuropathy (SFN) is a major cause of painful burning, numbness and tingling in feet/hands. SFN often precedes diagnosis of diabetes a.k.a. “impaired glucose tolerance neuropathy.” Diagnostic efficiency of skin punch biopsy is ≈88%. Findings on routine nerve conduction studies and electromyography are typically normal (large fiber only). Management of SFN should involve controlling the symptoms and aggressively treating the underlying cause.

Current treatments of peripheral neuropathy only target the positive (painful) symptoms. 25% of patients receive no treatment. 53.9% of patients receive opioids and 39.7% of patients receive NSAIDs for their pain. Other treatments include 21.1% SSRI's/SNRI's; 11.3% tricyclic antidepressants (TCAs), and 11.1% anticonvulsants.

Table A below provides Treatment Options for DPNP:

| Class | Therapy | Dose (mg/day) |
|---|---|---|
| Aldose reductase inhibitors | Epalrestat | Only licensed in Japan |
| Angiotensin-converting | Trandalopril | More studies needed |
| enzyme inhibitors | | |
| Tricyclic antidepressants | Amitriptyline | 20-150 |
| | Imipramine | 25-150 |
| SNRIs | Duloxetine* | 60-120 |
| Anticonvulsants | Gabapentin | 900-3600 |
| | Lamotrigine | 200-400 |
| | Carbamazepine | 200-600 |
| | Pregabalin* | 300-600 |
| Antiarrhythmics | Mexilitene | Up to 900 |
| Opioids | Tramadol | 50-400 |

*FDA Approved. Adapted from Tavakoli M. and Malik R. Expert Opin Pharmacother. 2008.

Current oral therapies for symptomatic treatment of peripheral neuropathy include: gabapentin (Neurontin), pregabalin (Lyrica), SNRI's such as duloxetine (Cymbalta). Local treatments are capsaicin and lidocaine creams and patches.

The only therapy that potentially treats and reverses the pathologic process of DPN is Metanx®, an oral prescription combination “medical food” of methyl folate, methyl cobalamim (B12), and 5-pyridoxal phosphate, B6.

No topical treatment currently exists for peripheral neuropathic conditions, of which diabetic peripheral neuropathy, DPN, is most prevalent that both alleviates “positive” symptoms (such as pain, tingling, and burning) but also reverses the neurpathic process, improving the “negative” symptoms of numbness and sensory loss. Topical lidocaine and capsaicin only affect “positive” symptoms by de-activating symptomatic damaged nerves.

In certain preferred embodiments of the present invention, the drug used to treat the neuropathic condition is a dopamine agonist. In certain embodiments, the drug is a dopamine agonist such as apomorphine (Apokyn®, APO-go®), pramipexole (Mirapexin®), ropinirole (Requip®), bromocriptine (Parlodel®), cabergoline (Cabaser®, Dostinex®), pergolide (Permax®, Celance®) rotigotine (Neupro®), dopamine, mixtures of any of the foregoing, or other dopamine agonists known to those skilled in the art.

In certain embodiments, the drug is a dopamine agonist such as apomorphine (Apokyn®, APO-go®), pramipexole (Mirapexin®), ropinirole (Requip®), bromocriptine (Parlodel®), cabergoline (Cabaser®, Dostinex®), pergolide (Permax®, Celance®) rotigotine (Neupro®), dopamine, mixtures of any of the foregoing, or other dopamine agonists known to those skilled in the art. One skilled in the art will appreciate that dopamine agonists other than apomorphine may be used in the formulations and methods of the present invention, and all such agents are meant to be encompassed by the term “dopamine agonists.” For example, such drugs include, but are not limited to, carbidopa (Sinemet®), dopamine agonists (Requip®, Rotigotine®, Mirapex®), COMT inhibitors (Entacapone®, Tocapone), rasagiline (Azilect®) (MAO inhibitors) and MAO-B inhibitors (Selegiline (Eldepryl®). Topical dopamine agonists (e.g., apomorphine, dopamine) as a compound which acts at dopamine, norepinephrine, and serotonin receptors) have also been found by the author to treat symptoms of neuropathic pain and other symptoms of nerve dysfunction, such as occurs in demyelination.

In certain preferred embodiments of the present invention, the drug used to treat the neuropathic condition is a drug which enhances conduction of damaged demyelinated nerves (e.g., a drug which blocks leaking potassium channels in damaged demyelinated nerves to improve conduction). In certain preferred embodiments, the drug is 4-aminopyridine, 3,4 diaminopyridine, tetraethylammonium (TEA), or combinations of any of the foregoing. 4-AP is particularly preferred.

In certain preferred embodiments of the present invention, the treatment (drug) comprises a combination of one or more dopamine agonists together with one or more drugs which enhance conduction of damaged demyelinated nerves (e.g., a drug which blocks leaking potassium channels in damaged demyelinated nerves to improve conduction), e.g., as set forth above.

In certain preferred embodiments of the present invention, the treatment may further comprise one or more additional drugs which are known to treat symptoms of neurologic conditions, including but not limited to neurotrophic agents which help nerve healing (methyl folate, methyl cobalamin, 5-pyridoxal phosphate, B1 benfotiamine, and stabilized R-Alpha Lipoic Acid (R-ALA); gabapentin; pregabalin; and a drug which provides neurotrophic support selected from the group consisting of methylcobalamin, L-methylfolate (vitamin B9), methylcobalamin (vitamin B12), pyridoxal 5′-phosphate (vitamin B6), and mixtures thereof; a drug capable of treating peripheral pain arising from the neurologic condition, the drug selected from the group consisting of an opioid analgesic, a non-steroidal anti-inflammatory agent (NSAID), a local anesthetic, and mixtures of any of the foregoing.

Disease modify agents which have been used for DPN include the following: glucose control, pancreas transplantation, administration of alpha lipoic acid (antioxidant) 600 mg intravenously or 1200-1800 mg orally, and neurotrophic support (L-methylfolate, pyridoxal 5′-phosphate (P5P), methylcobalamin (Me-Cbl) in amounts of 2.8 mg, 25 mg and 2 mg, respectively, twice daily). Such agents may be incorporated into the formulations of the present invention and/or the method of treatment. The only therapy that potentially treats and reverses the pathologic process of DPN is Metanx®, an oral prescription combination “medical food” of methyl folate (vitamin B9), methyl cobalamim (vitamin B12), and 5-pyridoxal phosphate (vitamin B6).

L-methylfolate increases nitric oxide synthesis, which improves blood flow to cutaneous nerves. It is the active form of folate necessary for neural function, and works with Methyl B12 to activate protein, DNA/RNA synthesis. It increases nitric oxide synthesis.

Methylcobalamin is the neurologically active form of vitamin B12. It is a methyl donor in DNA metabolism, up-regulate gene transcription for peripheral nerve repair & regeneration, and enhances protein metabolism in Schwann Cells.

Pyridoxal 5′-phosphate is the active form of B6, and is necessary for neural function. It may inhibit effects of advanced glycation end products.

When combined in a topical preparation with neurotrophic agents which help nerve healing (methyl folate, methyl cobalamin, 5-pyridoxal phosphate, B1 benfotiamine, and stabilized R-Alpha Lipoic Acid (R-ALA), there occurs both symptomatic and “disease modifying” nerve repair therapy.

Further drugs which have been used in the treatment of peripheral neuropathy (albeit not topically or in accordance with the presently disclosed methods) may be incorporated into the formulations and methods of the present invention. Such drugs include gabapentin (Neurontin), pregabalin (Lyrica), serotonin-norepinephrin reuptake inhibitors (SNRI's, such as duloxetine (Cymbalta), Venlafaxine, Milnacipran, Desvenlafaxine, Desvenlafaxine succinate, Duloxetine hydrochloride, Venlafaxine Hydrochloride, Milnacipran hydrochloride, and mixtures thereof) and selective serotonin reuptake inhibitors (SSRI's, such as Fluoxetine, Sertraline, Paroxetine, Citalopram, Escitalopram, Dapoxetine, Seproxetine, Zimelidine, Mesembrine, and mixtures thereof).

Other agents which may be incorporated into the formulations and treatments of the present invention include capsaicinoids such as capsaicin, and one or more local anesthetics (e.g., lidocaine, Procaine Benzocaine, Chloroprocaine, Cocaine, Cyclomethycaine, Dimethocaine/Larocaine, Piperocaine, Propoxycaine, Procaine/Novocaine, Proparacaine, Tetracaine/Amethocaine, Bupivacaine, Cinchocaine/Dibucaine, Etidocaine, Levobupivacaine, Lidocaine/Lignocaine, Mepivacaine, Prilocaine, Ropivacaine, Trimecaine, and mixtures and derivatives of any of the foregoing).

Current oral therapies for symptomatic treatment of peripheral neuropathy include: gabapentin (Neurontin), pregabalin (Lyrica), SNRI's such as duloxetine (Cymbalta). Local treatments are capsaicin and lidocaine creams and patches.

The drug formulations useful in the present invention may be in a form selected from a topical formulation (e.g, a cream, ointment or gel); a transdermal device; or an implantable or injectable formulation.

In certain preferred embodiments, the active agent(s) is included in a topical formulation further comprising one or more pharmaceutically acceptable excipients that aid in the absorption of the active agent(s) when a unit dose of the formulation is applied topically to the headache region of the human patient.

In certain other embodiments, the active agent(s) provide a localized effect and/or reduced side effects.

Certain embodiments of the invention are directed to a method of treatment, comprising delivering a drug(s) through peripheral neuro-affective therapy by application as a cream/gel or a sustained release patch applied at the site of the injury, or via administration under the skin at the site of the injury via an implantable or injectable drug formulation or device.

In certain embodiments, the method further provides for a therapeutically effective treatment through transdermal peripheral neuro-affective (TPNA) therapy by application of a drug(s) as a cream/gel or a sustained release patch applied at the site of the injury.

The present peripheral TRNA delivery differs from traditional therapy (whether oral, injection, nasal spray, inhalation, or rectal) in that it has no reliance on the systemic or cerebral blood flow. Nor does it require therapeutic blood levels of drug. These latter factors are responsible for systemic and CNS side-effects as drug is delivered to areas not intended to be affected in the therapeutic process. Transdermal systemic delivery by patch differs significantly in its reliance on a drug concentration gradient for absorption into the systemic capillary and venous blood. TRNA therapy is unaffected by dermal vessels or systemic blood flow. It relies solely on the function of the free nerve endings of cutaneous nerves and their connections at the point of application of compounded drug.

“Traditional” transdermal drug delivery by patch and TPNA are both “transdermal” in that in both, drug penetrates the skin (epidermis) for eventual clinical effect. The difference lies in the fact that in “traditional” transdermal patch therapy, drug enters the systemic circulation through a concentration gradient and establishes a therapeutic drug blood level. Although measuring a blood level gives assurance drug is being taken or delivered systemically, allowing for checking compliance, it is also the source of undesirable side-effects and drug interactions. Of necessity, with systemic transdermal patch therapy, drug applied to the skin surface must be absorbed through the small vessels in the dermis for eventual presence in the systemic venous blood for measurement of drug level. With the proposed TPNA therapy, drug need only be available at the free nerve endings under the epidermis. No concentration gradients or systemic blood levels are necessary. Drug delivery is unaffected by cardiac output or cerebral blood flow factors.

Thus, in certain embodiments, the methods and formulations of the invention deliver an amount of drug (e.g., dopamine agonist) in the TPNA therapy that would provide subtherapeutic plasma levels if administered orally, but which is therapeutically effective when administered via TPNA therapy.

It is hypothesized by the inventor that a principal reason TPNA therapy is rapid in the onset of clinical effect (e.g., less than about 10-15 minutes with topical apomorphine) is that it operates through an “electro-chemical” process. Active drug compounded in an appropriate dermal penetration enhancing medium acts at free nerve endings, changing the neurochemistry of receptors at the neural synapse: apomorphine (dopamine and norepinephrine agonist), increasing dopamine and norepinephrine levels and improving neural transmission. After a point of receptor stimulation, neural (electrical) impulses are generated back to neuronal cell bodies residing in the spinal cord and brainstem: “afferent feed-back”. The nervous system functions through neurons generating electrical impulses and the release of neurochemicals/neurotransmitters (serotonin, norepinephrine, dopamine, and acetylcholine, being the major ones) at neural receptor sites called “synaptic clefts”. Accordingly, the process in TPNA therapy may be considered analogous to an electrical capacitor discharging to perform a function, such as turning on a light switch. Viewed from this perspective, the rapid onset of clinical effect observed in TPNA therapy makes sense. This hypothesis is provided for (possible) explanatory purposes only and is not meant to in any way limit the scope of the present invention.

Alternatively, rapid as well as prolonged clinical effect may be achieved by a sustained-release dermal system employing the principles of TPNA therapy through patch application at the skin at the site of the injury. The location is critical in TPNA therapy, whereas, with a transdermal systemic patch, location is irrelevant.

The inventor has previously reported on the use of compounded topical apomorphine as a cream for the treatment of peripheral and spinal pain conditions. The proposed mechanism of action is based on the effect of apomorphine on nociceptive cutaneous nerve endings and spinal nerve roots which transmit pain impulses to the spinal cord dorsal horn and brain processing areas for interpretation as pain and other symptoms. Apomorphine possesses agonist (enhancing/stimulating) action on serotonin (5-HT), norepinephrine (NE), and dopamine (DA) receptors. 5-HT and NE are known to play a major role in the attenuation of pain signals from peripheral sites of injury which produce nociceptive (pain) input.

Endorphins and enkephalins produced by the body (endogenous opioids), or provided as opiate drugs and narcotics (exogenous opioids) also alleviate pain by inhibiting pain signals from sites of nerve injury. GABA, gamma butyric acid, a chemical produced by the body is also a pain inhibitor. On the other hand, substances that are excitatory to and enhance the pain process are: NMDA-glutamate, nitric oxide, and Substance P.

Studies indicate there are opioid receptors on free nerve endings of skin which may be affected by topically applied compounded opiate drugs to provide pain relief. The presence of opioid receptors and of endorphins in the central nervous system has been known since the 1970's. These play roles in pain processing and interpretation by the brain. However, with the knowledge these receptors also exist on skin nerve endings suggest they may be activated topically. Indeed, studies by Tennant indicate compounded morphine (in cold cream) when applied locally to the skin at areas of pain provided relief. Further, morphine levels were not detected in blood or urine, indicating lack of systemic absorption. It was concluded morphine acted directly on opiate receptors on nerve endings to produce pain relief and not systemically; that is, through the blood and then acting on the brain.

Topical Neuro-Affective Drug Therapy is the means by which a “neuro-active” drug (one acting to alter nerve function either “peripherally” in the extremities or spine; or “centrally,” in the brain, to treat symptoms attributable to the nervous system—pain, mood disorders, seizures, movement disorders such as Parkinson's disease or essential tremor, etc.) is applied to the skin as a compounded preparation to enhance penetration to the level of the free nerve-endings below the skin surface to exert therapeutic effects. Previously described has is the “nuchal” (back of the neck at the hairline, or “BONATH”) application of triptans (Migraderm using sumatriptan) for migraine headache, tizanidine (Tizoderm) for cervical spasm and tension headache; and, apomorphine for the Parkinson's disease symptoms of tremor, rigidity, speech difficulties, and other “non-motor” symptoms. Compounded morphine sulfate, and more recently, tramadol have been applied at the nuchal region for the relief of both local peripheral and generalized pain with good results. Pain reduction in treated patients has been in the range of 30-60%. Patients have used these preparations on a regular basis with continued benefit. In fact, a number have been able to completely taper off their oral narcotics, using the nuchal delivery as their only method of pain therapy. Many systemic side-effects of narcotics such as lethargy, fatigue, cognitive slowing, and depression have significantly diminished in these patients, improving quality of life. Further, no significant narcotic withdrawal symptoms were noted.

Applied at low doses and as a cream rubbed onto the skin to affect the cutaneous (skin) nerve endings without absorption into the subcutaneous soft tissue and blood vessels, one may achieve pain relief from topical opiate drugs without the general systemic effects of addiction, tolerance (a pharmacological term for the need for progressively higher doses of drug for same effect), and CNS suppression. If these latter undesirable side-effects of opioid drugs could be minimized or prevented by topical neuro-affective therapy, their negative impact on society would also be curtailed. Opiates delivered in this manner could be prescribed for de nouveau pain patients to prevent addiction; and, to those already on chronic use, to conceivably reduce the negative effects of high dose chronic use by traditional methods.

Topical neuro-affective therapy, via both peripheral nerve activation and at the nuchal region provides an alternative method of using pain relieving drugs with significant potential harm in a safer way with diminished side-effects. This technology may be applied to the opioid class of potent narcotics such as morphine as well as to opiate receptor agonists like tramadol and the SNRI class of drugs which are also used to treat pain—venlafaxine (Effexor®), duloxetine (Cymbalta®), milnaciprin (Savella®), and the like.

One skilled in the art having the benefit of the information contained herein will appreciate that there are many classes of drugs which would be useful for topical peripheral de-afferentation therapy.

In certain embodiments, the formulation includes an opioid analgesic (opioid agonist), administered to the area(s) in an effective amount to provide the desired analgesic effect. Opioids, also known as opioid agonists, are a group of drugs that exhibit opium or morphine-like properties. The opioids are employed primarily as moderate to strong analgesics, but have many other pharmacological effects as well, including drowsiness, respiratory depression, changes in mood and mental clouding without a resulting loss of consciousness. Opioids act as agonists, interacting with stereospecific and saturable binding sites in the brain and other tissues. Endogenous opioid-like peptides are present particularly in areas of the central nervous system that are presumed to be related to the perception of pain; to movement, mood and behavior, and to the regulation of neuroendocrinological functions. Opium contains more than twenty distinct alkaloids. Morphine, codeine and papaverine are included in this group.

Opioid analgesics which are useful in the present invention include all opioid agonists or mixed agonist-antagonists, partial agonists, including but not limited to alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, mixtures of any of the foregoing, salts of any of the foregoing, and the like, the term “opioid” for the purposes of the present invention is considered to encompass synthetic entities with morphine-like actions, and all exogenous substances that bind stereo-specifically to any of several subspecies of opioid receptors and produce agonist actions.

In certain preferred embodiments, the opioid agonist or analgesic is selected from the group consisting of tramadol, hydrocodone, morphine, hydromorphone, oxycodone, codeine, levorphanol, meperidine, methadone, or salts thereof, or mixtures thereof. The dose for MS has been, e.g., 2.5-5.0 mg and for tramadol, 40-80 mg. In certain preferred embodiments, the opioid agonist is hydrocodone. Equianalgesic doses of these opioids, in comparison to a 15 mg dose of hydrocodone, are set forth in Table 1 below:

TABLE 1
Equianalgesic Doses of Opioids

| Opioid | Calculated Dose (mg) |
|---|---|
| Oxycodone | 13.5 |
| Codeine | 90.0 |
| Hydrocodone | 15.0 |
| Hydromorphone | 3.375 |
| Levorphanol | 1.8 |
| Meperidine | 135.0 |
| Methadone | 9.0 |
| Morphine | 27.0 |

The invention disclosed herein is meant to encompass all pharmaceutically acceptable salts thereof of the disclosed opioid agonists. Some of the opioid agonists encompassed within the present invention may contain one or more asymmetric centers and may thus give rise to enantiomers, diastereomers, and other stereoisomeric forms. The present invention is also meant to encompass the use of all such possible forms as well as their racemic and resolved forms and mixtures thereof. When such compounds contain olefinic double bonds or other centers of geometric asymmetry, and unless specified otherwise, it is intended to include both E and Z geometric isomers. All tautomers are intended to be encompassed by the present invention as well. As used herein, the term “stereoisomers” is a general term for all isomers of individual molecules that differ only in the orientation of their atoms is space. It includes enantiomers and isomers of compounds with more than one chiral center that are not mirror images of one another (diastereomers). The term “chiral center” refers to a carbon atom to which four different groups are attached. The term “enantiomer” or “enantiomeric” refers to a molecule that is nonsuperimposeable on its mirror image and hence optically active wherein the enantiomer rotates the plane of polarized light in one direction and its mirror image rotates the plane of polarized light in the opposite direction. The term “racemic” refers to a mixture of equal parts of enantiomers and which is optically inactive. The term “resolution” refers to the separation or concentration or depletion of one of the two enantiomeric forms of a molecule.

In certain preferred embodiments, the opioid analgesic is selected from tramadol, morphine, oxycodone, oxymorphone, hydromorphone, hydrocodone, pharmaceutically acceptable salts thereof, and mixtures thereof. In certain preferred embodiments, the opioid analgesic is a non-narcotic opioid such as tramadol. In certain preferred embodiments, tramadol is applied topically as the hydrochloride salt in an amount from about 20 to about 40 mg. In certain preferred embodiments, morphine is applied topically as its sulfate salt in an amount from about 2.5 mg to about 5 mg. In other preferred embodiments a narcotic or non-narcotic opioid analgesic is used in accordance with the present invention in a equianalgesic dose, e.g., as known to those skilled in the art and/or as set forth in Table 1 above.

In certain embodiments, the formulation includes one or more non-steroidal anti-inflammatory agents (NSAIDs). The formulation may include a therapeutically effective amount of cyclooxygenase-1 (COX-1) and cycloxygenase-2 (COX-2) inhibitors (collectively known as NSAIDs), such as Aspirin (acetylsalicylic acid), Celecoxib, Dexdetoprofen, Diclofenac, Diflunisal, Etodolac, Etoricoxib, Fenoprofen, Firocoxib, Flurbiprofen, Ibuprofen, Indomethacin, Ketoprofen, Ketorolac, Licofelone, Lornoxicam, Loxoprofen, Lumiracoxib, Meclofenamic acid, Mefenamic acid, Meloxicam, Nabumetone, Naproxen, Nimesulide, Oxaporozin, Parecoxib, Piroxicam, Rofecoxib, Salsalate, Sulindac, Tenoxicam, Tolfenamic acid, Valdecoxib, or any combination thereof.

Equianalgesic doses of a wide variety of NSAIDs are generally known and understood to those having ordinary skill in the art. For example, equianalgesic oral doses of certain NSAIDs and acetaminophen may be as follows: acetaminophen 975 mg; aspirin 975 mg; ibuprofen 400-600 mg; magnesium salicylate 650 mg; naproxen 250-275 mg; naproxen sodium 275 mg; carprofen 100 mg; choline magnesium trisalicylate 1000-1500 mg; choline salicylate 870 mg; diclofenac 50 mg; diflunisal 500 mg; etodolac 200-400 mg; fenoprofen 300-600 mg; ketoprofen 25-60 mg; ketorolac 10 mg; meclofenamate 50-100 mg; mefenamic acid 250 mg; sodium salicylate 325-650 mg.

In certain embodiments, the formulation includes an SNRI (serotonin-norepinephrine reuptake inhibitor). SNRIs are used in the treatment of major depression and other mood disorders, anxiety disorders, obsessive-compulsive disorder (OCD), attention deficit hyperactivity disorder (ADHD), chronic neuropathic pain, fibromyalgia syndrome (FMS), and for the relief of menopausal symptoms. SNRIs act upon and increase the levels of two neurotransmitters in the brain, i.e., serotonin and norepinephrine. This can be contrasted with selective serotonin reuptake inhibitors (SSRIs), which only act on serotonin. Non-limiting examples of SNRIs which may be used in the methods of the present invention include venlafaxine, desvenlafaxine, duloxetine, milnacipran, levomilnacipran, sibutramine, bicifadine, SEP-227162, and LY 2216684. Generally, it is contemplated that the unit dose initially applied at a location(s) of the SNRI will be about one-half of the starting oral dose of that drug. For example, that would translate to an initial topical dose of about 37.5 mg for venlafaxine, an initial topical dose of about 15 mg for duloxetine, and an initial topical dose of about 7.5 mg for milnacipran.

The topical application of various combinations of compounds currently used orally to treat neuropathic conditions works faster and better than their oral counterparts. Further, multiple drug combinations may be used to provide synergistic effect without concern for drug-drug interactions and systemic side effects.

In preferred embodiments, the average time to notation of improved neuropathic pain, less numbness, and improved blood flow to the affected area is from about 10 to about 15 minutes.

In certain embodiments, the formulations described herein are fast acting. For example, the symptoms associated with neurologic conditions begin to be or are relieved within about 2 hours, preferably within about 5 minutes to about 2 hours, within about 5 minutes to about 1 hour and most preferably within about 5 minutes to about 30 minutes after application of the formulation. In certain preferred embodiments, the formulations of the present invention provide relief or begin to provide efficacy within from less than 1 minute to about 2 hours, from about 1 minute to about 2 hours, and most preferably from about 1 minute to about 15 minutes.

In preferred embodiments, the formulation is applied from about 1 to about 4 times per day at the affected areas of the patient. It has been found that 2-3 applications a day are sufficient to control symptoms throughout the day and night in many patients.

With topical application of these neurotrophic agents, evidence of nerve repair (improved sensation and movement) are evident within 2-3 weeks as opposed to months with the oral route (i.e., via administration of Metanx®). There is nothing currently available which treats peripheral neuropathic conditions, such as DPN, as effectively and as rapidly. Not only are symptoms alleviated; but, there is repair of nerve damage.

The application of the compounded cream is to the areas of involvement: feet, hands, and areas of nerve compression: wrist, elbow, inguinal ligament, fibula head.

In applying peripherally at the exact area of nerve dysfunction, the compounds are delivered where they need to work, as opposed to oral intake of the same. Further, since blood flow is already compromised in these conditions, oral drugs have a more difficult time getting to the pathologic areas. This problem is by-passed with topical therapy where the compounds easily reach the affected nerves and blood vessels.

The methods of the present invention may also, if desired, involve pre-treatment of the skin with an enhancer to increase the permeability of the skin to the applied drug. The methods of the present invention may include pre-treatment or “prepping” of the skin area with a substance that opens up the skin pores. Additionally, the methods of the present invention may include, if desired, pre-treatment or “prepping” of the skin with an alcohol swab or the like to rid the area of dirt, make-up, oil, and the like, prior to application of the drug.

### FORMULATIONS

All currently approved therapies for the conditions described above reach the central nervous system through the systemic circulation. Cerebral blood flow to brainstem structures is through the posterior circulation, via the vertebral and basilar arteries and their branches. In view of the undesirable side-effects associated with this form of drug delivery to the brain, it makes sense that targeted regional delivery to the brainstem is sought. Topical delivery of currently used drugs compounded in an appropriate “dermal penetration enhancer” and applied in cream/gel form or as a sustained-release patch at the posterior cervical region (back of the neck) at the hairline is such a method. Lipoderm® is an example of an effective commercially available compounding medium. However, one skilled in the art will recognize that topical carriers meeting the specific chemical requirements of an individual drug can be formulated for maximum efficiency in topical delivery.

The formulations of the present invention are prepared such that the drug(s) may be delivered acutely as single dose applications as cream/gel/ointment or as a sustained release topical patch, depending on the condition treated and associated symptom complex in the individual patient. The critical point, again, is in the location of the application: at the back of neck at the hair-line for access to posterior cervical afferents with free nerve endings under the surface of the skin. Through feedback connections with vagal and trigeminal afferent systems, this results in ultimate effect on brainstem structures.

By virtue of the method of treatment described herein, the disease state/condition to be treated may be treated much faster and more effectively than such prior art modes of administration.

In certain embodiments of the present invention, the method of treating a human patient comprises applying a topical formulation which comprises a drug suitable for topical administration, which is useful for the treatment of a disease state or condition treatable via the topical brainstem afferent stimulation (de-afferentation) drug therapy described herein.

The methods of the present invention may also, if desired, involve pre-treatment of the skin with an enhancer to increase the permeability of the skin to the applied drug. The methods of the present invention may include pre-treatment or “prepping” of the skin area with a substance that opens up the skin pores. Additionally, the methods of the present invention may include, if desired, pre-treatment or “prepping” of the skin with an alcohol swab or the like to rid the area of dirt, make-up, oil, and the like, prior to application of the drug.

In certain embodiments, the topical formulation of the present invention comprises a drug in an amount which is therapeutically effective when administered topically at the at the back of neck at the hair-line for access to posterior cervical afferents with free nerve endings under the surface of the skin, but which provides a plasma concentration which is subtherapeutic if orally administered.

In certain embodiments, by applying the formulation of the present invention comprising a dose of drug at the back of neck at the hair-line for access to posterior cervical afferents with free nerve endings under the surface of the skin, it may be possible for the use of lower doses of drug or faster relief of the headache than if applied to the trunk or limbs of a human patient, and the lower plasma levels of drug which result from lower doses may thereby reduce unwanted side effects of the drug.

The topical formulations of the present invention (e.g., ointment, gel, cream, or the like), must be suitable for topical administration of a drug, i.e., must contain pharmaceutically acceptable excipients compatible with application to the skin tissue, and may optionally contain a sufficient amount of an enhancer composition as described hereinafter.

In certain embodiments, in addition to the drug (e.g., dopamine agonist), the topical formulations and/or transdermal therapeutic systems of the present invention may include at least one adjuvant such as a penetration enhancer, anti-oxidant, stabilizer, carrier, or vehicle. Additionally or alternatively, the present invention may include the application of electric current (iontophoresis) for enhancing permeation of the dopamine agonist.

In certain embodiments, the topical formulations comprising a drug in an ointment, gel, cream or the like, will typically contain on the order of about 0.001 to about 80% by weight, preferably 0.01 wt. % to 50 wt. % drug, and about 0 wt. % to about 50.0 wt. %, preferably from about 1 wt. % to about 30 wt. % of a permeation enhancer composition, with the remainder of the composition comprising a carrier or vehicle. In certain preferred embodiments, the drug is included in a cream or gel or ointment in a concentration of, e.g., 1 mg drug/ml of carrier (e.g., Lipoderm). However, it is to be understood that one skilled in the art can increase the amount of carrier or change the carrier and maintain or improve efficacy of the topical formulation for TRNA therapy.

In certain embodiments, the topical formulations comprising a dopamine agonist in an ointment, gel, cream or the like, will typically contain on the order of about 0.001 to about 80% by weight, preferably 0.01 wt. % to 50 wt. % dopamine agonist, and about 0 wt. % to about 50.0 wt. %, preferably from about 1 wt. % to about 30 wt. % of a permeation enhancer composition, with the remainder of the composition comprising a carrier or vehicle. In certain preferred embodiments, the dopamine agonist is apomorphine and is included in a cream or gel or ointment in a concentration of, e.g., 1 mg drug/ml of carrier (e.g., Lipoderm). However, it is to be understood that one skilled in the art can increase the amount of carrier or change the carrier and maintain or improve efficacy of the topical formulation for TRNA therapy. In certain preferred embodiments, the drug is applied as a unit dose at the BONATH in immediate release form (e.g., cream, ointment or gel) for acute treatment with a dopamine agonist as would be beneficial to a person suffering from, e.g., Parkinson's disease or impotence/male erectile dysfunction. In such instances, it is preferred that the concentration of dopamine agonist included in the unit dose is from about 0.25 mg to about 4 mg, based on apomorphine, or an therapeutically equivalent amount of another dopamine agonist as described herein.

Suitable permeation enhancers may also be included in the formulations. Such enhancers include, but are not limited to, dimethylsulfoxide (DMSO), N,N-dimethylacetamide (DMA), decylmethylsulfoxide (C10 MSO), polyethylene glycol monolaurate (PEGML), propylene glycol (PG), PGML, glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, and the like. The permeation enhancer may also be a vegetable oil as described in U.S. Pat. No. 5,229,130 to Sharma. Such oils include, for example, safflower oil, cotton seed oil and corn oil.

Additional enhancers for use in conjunction with the present invention are lipophilic compounds having the formula [RCOO]n R′, wherein n is 1 or 2 and R is C1-C16 alkyl optionally substituted with 1 or 2 hydroxyl groups, and R′ is hydrogen or C1-C16 alkyl optionally substituted with 1 or 2 hydroxyl groups. Within this group, a first subset of compounds are represented by the formula [CH3 (CH2)m COO]n R′ in which m is an integer in the range of 8 to 16, n is 1 or 2, and R′ is a lower alkyl (C1-C3) residue that is either unsubstituted or substituted with one or two hydroxyl groups. Preferred enhancers within this group include an ester which is a lower alkyl (C1-C3) laurate (i.e., m is 10 and n is 1) such as “PGML”. It will be appreciated by those skilled in the art that the commercially available material sold as “PGML” is typically although not necessarily a mixture of propylene glycol monolaurate itself, propylene glycol dilaurate, and either propylene glycol, methyl laurate, or both. Thus, the terms “PGML” or “propylene glycol monolaurate” as used herein are intended to encompass both the pure compound as well as the mixture that is typically obtained commercially. Also within this group is a second subset of compounds, namely, esters of fatty alcohols represented by the formula CH3 (CH2)m-O—CO—CHR1 R2, in which R1 and R2 are independently hydrogen, hydroxyl, or lower alkyl (C1-C3), and m is as above. Particularly preferred enhancers within this group are lauryl lactate and myristyl lactate. In addition, a third subset of compounds within this group are analogous fatty acids, i.e., acids having the structural formula CH3 (CH2)m COOH where m is as above. A particularly preferred acid is lauric acid.

Other enhancer compositions are wherein a lipophilic compound as just described, particularly PGML is combined with a hydrophilic compound, such as a C2-C6 alkanediol. One preferred hydrophilic enhancer within this group is 1,3-butanediol. Such enhancer compositions are described in detail in PCT Publication No. WO 95/05137, published Feb. 23, 1995, herein incorporated by reference. Another hydrophilic enhancer that may be included in these compositions is an ether selected from the group consisting of diethylene glycol monoethyl ether (Transcutol) and diethylene glycol monomethyl ether. Such enhancer compositions are described in detail in U.S. Pat. Nos. 5,053,227 and 5,059,426 to Chiang et al., the disclosures of which are herein incorporated by reference.

Other enhancer compositions may include mixture or combinations of any of the aforementioned enhancers, and the like.

In certain embodiments the topical formulation may include at least one water-insoluble, pharmacologically approved, alkyl cellulose or hydroxyalkyl cellulose, and the like. Alkyl cellulose or hydroxyalkyl cellulose polymers for use in this invention include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross linking agent may be used to modify the polymer's characteristics. For example, esters such as dibutyl or diethyl phthalate, amides such as diethyldiphenyl urea, vegetable oils, fatty acids and alcohols such as acid oleic and myristyl may be used in combination with the cellulose derivative.

In certain embodiments, the topical formulation may further include hydrocarbons such as liquid paraffin, vaseline, solid paraffin, microcrystalline wax, etc.; higher aliphatic alcohols such as cetyl alcohol, hexadecyl, alcohol, stearyl alcohol, oleyl alcohol, etc.; esters of higher fatty acids with higher alcohols such as beeswax, etc.; esters of higher fatty acids with lower alcohols such as isopropyl myristate, isopropyl palmitate, etc.; vegetable oils, modified vegetable oils, hydrous lanolin and its derivative, squalene, squalane; higher fatty acids such as palmitic acid, stearic acid, etc. and the like.

In certain embodiments, the topical formulation may further include emulsifiers and dispersing agents which include, for example, anionic, cationic and nonionic surfactants. Nonionic surfactants are preferred because of their low levels of irritation to skin. Typical of nonionic surfactants are fatty acid monoglycerides such as glyceryl monostearate, etc.; sorbitan fatty acid esters such as sorbitan monolaurate, etc.; sucrose fatty acid esters; polyoxyethylene fatty acid esters such as polyoxyethylene stearate, etc.; and polyoxyethylene higher alcohol ethers such as polyoxyethylene cetyl ether, polyoxyethylene oleyl ether, etc.

In certain preferred embodiments, the topical TRNA formulation is aqueous-based.

In certain embodiments of the present invention, the topical formulation may include a gelling agent such as methylcellulose, ethylcellulose, hydroxyethylcellulose, hydroxypropyl-cellulose, hydroxypropylmethylcellulose, carboxymethylcellulose, carbomer, and the like. Examples of pharmaceutical compositions which rely upon an aqueous gel composition as a vehicle for the application of a drug are U.S. Pat. Nos. 4,883,660; 4,767,619; 4,511,563; 4,861,760; and 5,318,780, the disclosures of which are herein incorporated by reference.

The topical formulation may further include one or more preservatives, stabilizers, or anti-oxidants.

Examples of preservatives that may be used in a formulation according to the present invention include, but are not limited to, bacteriostatic compounds and other preservatives suitable for topical administration including various alcohols, sorbic acid and salts and derivatives thereof, ethylenediamine, monothioglycerol, and thimerosal.

Examples of stabilizers that may be present in a formulation according to the present invention include pH buffers suitable for topical administration, complexing agents, chelating agents and the like.

Examples of anti-oxidants that may be used in a formulation according to the present invention include ascorbic acid and its derivatives, e.g., ascorbyl palmitate, as well as butylated hydroxyanisole, butylated hydroxytoluene, sodium bisulfite, sodium metabisulfite, and others.

Other adjuvants that may be included in the drug formulation include carriers, tackifiers, pigments, dyes, and other additives that do not adversely affect the mechanical or adhesive properties of the formulation.

“Carriers” or “vehicles” as used herein refer to carrier materials suitable for transdermal drug administration, and include any such materials known in the art, e.g., any liquid, gel, emulsion, solvent, liquid diluent, solubilizer, or the like, which is nontoxic and which does not interact with other components of the composition in a deleterious manner. The term “carrier” or “vehicle” as used herein may also refer to stabilizers, crystallization inhibitors, dispersing agents or other types of additives useful for facilitating transdermal drug delivery. It will be appreciated that compounds classified as “vehicles” or “carriers” may sometimes act as permeation enhancers, and vice versa, and, accordingly, these two classes of chemical compounds or compositions may sometimes overlap.

Carrier materials suitable for use in the instant compositions include those well-known for use in the cosmetic and medical arts as bases for ointments, lotions, salves, aerosols, suppositories and the like. Suitable carriers include, for example, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers herein include for example alcohols, including both monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

In certain preferred embodiments of the present invention where it is desired that the drug (.e.g., dopamine agonist) is administered chronically, the formulations of the present invention may be formulated as a transdermal delivery system (also referred to herein as a transdermal therapeutic system) such as a transdermal patch, a transdermal plaster, a transdermal disc, iontophoretic transdermal device, or the like. Such formulations are recognized by those skilled in the art as providing a release of drug and absorption into the skin of the patient in a sustained manner over an extended period of time (e.g., 1-7 days). In such embodiments of the present invention, the transdermal delivery system comprises, e.g., a dopamine agonist contained in a reservoir or a matrix, and an adhesive which allows the transdermal patch to adhere to the skin, allowing the passage of the active agent from the transdermal patch through the skin of the patient. In preferred embodiments, the transdermal patch is applied topically at the back of the neck at the hairline (“BONATH”) so as to achieve topical regional neuro-affective therapy (“TRNA THERAPY”) as described herein. In embodiments in which the drug is contained in a transdermal patch, it is contemplated that the drug will be absorbed more slowly and the transdermal patch will provide a sustained release and prolonged therapeutic effect, as compared, e.g., to a cream or ointment intended to provide an immediate release of the drug and rapid onset of the TRNA therapy.

In certain embodiments, the transdermal delivery devices, as well as other transdermal delivery systems in accordance with the invention can be made in the form of an article such as a tape, a patch, a sheet, a dressing or any other form known to those skilled in the art. Generally the device will be in the form of a patch of a size suitable to deliver a unit dose of serotonin agonist through the skin. The drug may be introduced into a transdermal therapeutic system in different forms (solid, in solution, in dispersion); it may also be microencapsulated.

In certain embodiments the present invention provides a transdermal therapeutic system comprising a drug (e.g., serotonin agonist) in an amount that would provide subtherapeutic plasma levels if administered orally, but is therapeutically effective when administered via transdermal delivery at the headache region.

A transdermal delivery system for use in accordance with the present invention can also be constructed with an enhancer composition and other ingredients described hereinabove with respect to the topical formulation. Preferably, the transdermal delivery system is formulated for the prolonged delivery of a drug (e.g., dopamine agonist) as would be beneficial to a person suffering from, e.g., Parkinson's disease or impotence/male erectile dysfunction. The targeted skin flux for delivery of a particular drug can be achieved by adjusting vehicle composition and vehicle loading, as well as by adjusting the surface area through which the compositions are administered to skin.

In certain preferred embodiments, the transdermal delivery system (e.g., patch) is formulated to deliver from about 4 mg to about 50 mg of the dopamine agonist per each 24 hours through the skin of the patient, based on apomorphine, or a therapeutically equivalent amount of a suitable alternative dopamine agonist as described herein. In embodiments in which the transdermal delivery system is intended to be applied to the skin at the BONATH for multiple days, the transdermal delivery system (e.g., patch) is formulated to provide a flux rate over the useful life of the system such that a similar amount (e.g., mean dose) is delivered on a daily basis until the system is removed and replaced with a fresh system.

The transdermal delivery system used in the present invention may be prepared, for example, in accordance with U.S. Pat. Nos. 5,069,909; 4,806,341; 5,026,556; 4,588,580; 5,016,652; 3,598,122; 4,144,317; 4,201,211; 4,262,003; and 4,379,454; all of which are incorporated herein by reference.

Additionally, the transdermal delivery system used in the present invention may be in accordance with U.S. Pat. No. 6,689,379, hereby incorporated by reference, which system is a matrix or reservoir system which comprises: at least one pharmaceutical active agent selected from the group consisting of basic pharmaceutical active agents and neutral pharmaceutical active agents (such as rivastigmine); and a pressure-sensitive adhesive comprising a polyacrylate polymer, wherein said polyacrylate polymer has a polyacrylate backbone containing monomer units selected from the group consisting of acrylic acid, methacrylic acid and ester derivatives of acrylic or methacrylic acid, and said monomer units comprise at least 50% (w/w) relative to a mean polymer mass of said polyacrylate polymer, a total amount of monomers selected from the group consisting of non-esterified acrylic acid and non-esterified methacrylic acid is 0.5 to 10.0% (w/w) relative to the mean polymer mass of said polyacrylate polymer, and the carboxyl groups of said non-esterified acrylic and methacrylic acid monomers are present stoichiometrically at 5 to 100% in the form of alkali salts or alkaline-earth salts, said salts being reaction products of a neutralization reaction of an alcoholic solution of an alkaline hydroxide or an alkaline-earth hydroxide with said acrylate polymer(s), or of a neutralization reaction of an alkali alcoholate or an alkaline-earth alcoholate with said acrylate polymer(s).

In certain embodiments, the dosage form can be a transdermal patch comprising a laminated composite for administering the drug (e.g., dopamine agonist) to an individual transdermally comprising: (a) a polymer backing layer that is substantially impermeable to the dopamine agonist; and (b) a reservoir layer comprising a water-base acrylate pressure-sensitive adhesive, 1 to 12% by weight serotonin agonist and 2 to 25% by weight of a permeation enhancer comprising propylene glycol monolaurate in combination with capric acid or oleic acid, wherein the skin contact area of the composite is 10 to 100 cm2.

The dosage form can be a transdermal patch comprising (a) a polar solvent material selected from the group consisting of C3-C4 diols, C3-C6 triols, and mixtures thereof; and (b) a polar lipid material selected from the group consisting of fatty alcohol esters, fatty acid esters, and mixtures thereof; wherein said polar solvent material and said polar lipid material are present in a weight ratio of solvent material:lipid material of from about 60:40 to about 99:1.

In certain embodiments, the dosage form also comprises a transdermal plaster comprising: (1) a film layer which comprises a polyester film of 0.5 to 4.9 microns thickness, 8 to 85 g/mm strength, respectively in the two directions intersecting substantially at right angles, 30 to 150% elongation, in the two directions intersecting substantially at right angles and an elongation ratio of A to B of 1.0 to 5.0, wherein A and B represent data in two directions intersecting at right angles, and A is greater than B, and wherein said polyester film comprises 0.01 to 1.0% by weight, based on the total weight of said polyester film, of solid fine particles in which (a) the average particle size is 0.001 to 3.0 microns, and (b) the average particle size is substantially not more than 1.5 times the thickness of said polyester film; and (2) an adhesive layer (a) which is composed of an adhesive containing said serotonin agonist and further wherein said adhesive layer (a) is laminated on said film layer over the surface in a 2 to 60 microns thickness.

In certain embodiments, the dosage form can be a transdermal disc comprising: (a) a backing layer which is substantially impervious to the dopamine agonist; and (b) a polymer matrix disc layer which is adhered to said backing layer and which has microdispersed therein said serotonin agonist, said polymer being bioacceptable and permitting said serotonin agonist to be transmitted for transdermal absorption, the dopamine agonist being stable in said polymer matrix.

In certain embodiments, the topical formulation or transdermal therapeutic system may further comprise another active ingredient in combination with the first drug (e.g., dopamine agonist), e.g., analgesics, antimimetics, psychopharmacologic agents, or sedatives.

The present invention is contemplated to encompass all transdermal formulations, e.g., the technologies described above, with the inclusion of a drug (e.g., dopamine agonist(s)), such that the administration of a drug useful for treatment of disease state or condition in humans via topical brainstem afferent stimulation (de-afferentation) therapy via topical administration. Therefore, modifications of the invention via, e.g., the choice and/or amount of drug are considered to be obvious variations of this disclosure and within the scope of the appended claims.

The present invention also contemplates the administration of the drug (e.g., dopamine agonist) directly below the skin to affect direct brainstem afferent stimulation to the free nerve endings under the epidermis. Such administration may be effected as an injection (e.g., subcutaneous injection) or implantation of the drug in immediate release or sustained release form. It will be appreciated by those skilled in the art that providing the drug in sustained release form and administering it in a suitable form below the skin may provide benefits, including less frequent administration (e.g., in chronic therapy).

In certain embodiments of the invention, the drug (e.g., dopamine agonist) can be formulated for controlled or sustained delivery at the BONATH via incorporation into a biocompatible and implantable polymer which can be in the form of microparticles or an implantable insert, or a liquid that forms a gel or colloid or a semi-solid after injection (thereby encapsulating the drug and allowing it to be released in a prolonged and controlled manner at the desired site). For chronic conditions (e.g., Parkinson's) or desired prolonged effect, it is contemplated that a drug depot or reservoir may be created under the skin at the BONATH, which then provides a sustained release of the drug in proximity to the desired nerve endings and which may be replenished or replaced at the end of the dosing interval. It is contemplated that such administrations of the drug (e.g., dopamine agonist) may provide a prolonged therapeutic effect for at least about 3 days, preferably at least about 7 days, or longer. Such formulations may be administered in certain embodiments as, for example, a subcutaneous depot.

Implants are placed subcutaneously by making an incision in the skin and forcing the implants between the skin and the muscle. At the end of their use, if not dissolved, these implants are surgically removed. U.S. Pat. No. 4,244,949, hereby incorporated by reference, describes an implant which has an outer matrix of an inert plastic such as polytetrafluoroethylene resin. Examples of this type of implantable therapeutic system are Progestasert IUD and Ocusert system. It is contemplated that such systems can be appropriately modified by one skilled in the art for use in conjunction with the present invention. A commercially available product, Norplant®, which is an implant having a core containing levonorgestrel as the active substance, and where the core it surrounded by a membrane of a silicone elastomer of poly(dimethylsiloxane) (PDMS). Another preparation of this kind is Jadelle®, in which the core is a poly(dimethylsiloxane) based matrix with levonorgestrel dispersed therein. The membrane is an elastomer made from PDMS and silica filler, which, besides giving necessary strength properties to the membrane, also retards the permeation of the active agent through the membrane. U.S. Pat. No. 3,854,480, hereby incorporated by reference, describes a drug delivery device, e.g. an implant, for releasing a drug at a controlled rate for a prolonged period of time. The device has a core of a matrix in which the drug is dispersed. The core is surrounded by a membrane that is insoluble in body fluids. The core matrix as well as the membrane are permeable to the drug by diffusion. The materials of the core and the membrane are chosen so that the drug diffuses through the membrane at a lesser rate than through the core matrix. Thus, the membrane controls the release rate of the drug. As a suitable polymer for the core matrix is mentioned poly(dimethylsiloxane) (PDMS), and as suitable polymers for the membrane are mentioned polyethylene and a copolymer of ethylene and vinyl acetate (EVA). It is contemplated that the above systems may be adapted by one skilled in the art to deliver the drug (e.g., dopamine agonists) in accordance with the present invention.

One device which may be adapted by one skilled in the art for use in the present invention is described in U.S. Pat. No. 5,968,542 (Tipton), hereby incorporated by reference, which describes a high viscosity liquid controlled delivery system as a medical or surgical device is provided that includes: (i) a non-polymeric, non-water soluble liquid carrier material (HVLCM) of viscosity of at least 5,000 cP at 37° C. that does not crystallize neat under ambient or physiological conditions; and, optionally, (ii) a substance to be delivered.

The pharmaceutical compositions suitable for injectable use in accordance with this invention include sterile aqueous solutions or dispersions and sterile powders or lyopholysates for the extemporaneous preparation of sterile injectable solutions or dispersions. The dosage forms must be sterile and it must be stable under the conditions of manufacture and storage. The carrier for injectable formulations is typically water but can also include ethanol, a polyol (for example, glycerol, propylene glycol and liquid polyethylene glycol), mixtures thereof, and vegetable oil.

Injectable formulations used in the present invention can also be formulated as injectable prolonged release formulations in which the active compound is combined with one or more natural or synthetic biodegradable or biodispersible polymers such as carbohydrates, including starches, gums and etherified or esterified cellulosic derivatives, polyethers, polyesters, polyvinyl alcohols, gelatins, or alginates. Such dosage formulations can be prepared for example in the form of microsphere suspensions, gels, or shaped polymer matrix implants that are well-known in the art for their function as “depot-type” drug delivery systems that provide prolonged release of the biologically active components. Such compositions can be prepared using art-recognized formulation techniques and designed for any of a wide variety of drug release profiles.

One example of a useful formulation which may be used in the methods of the present invention for providing a prolonged duration of action is described in U.S. Pat. No. 7,332,503 (Wikstrom, et al.), hereby incorporated by reference. Therein, apomorphine derivatives and the physiologically acceptable salts thereof as well as formulations thereof are described which provide a prolonged duration of action. The apomorphine pro-drugs can be suspended (as a neat oil or as crystals, or dissolved in a suitable and pharmaceutically acceptable solvent (e.g. water, ethanol, DMSO, i-PrOH or benzylbenzoate)) in a pharmaceutically acceptable depot oil (e.g. viscoleo, sesame oil or olive oil) and injected subcutaneously or intramuscularly with a syringe or a “pen injector”. Alternatively, these drugs may, in a suitable composition and with a suitable vehicle (penetration enhancer), be applied to a patch for transdermal administration. The composition could include also a local anesthetic (e.g. lidocaine) to avoid injection pain, in particular at intramuscular injections. In one embodiment, the composition is in the form of a patch or an ointment for transdermal administration. The patch or ointment preferably also comprises stabilizers, solubilizers and permeation activators to facilitate the passage of the active principle through the skin. In another preferred embodiment, the composition is in the form of a depot preparation for subcutaneous or intramuscular administration comprising an apomorphine derivative or the physiologically acceptable salt thereof dissolved or suspended in an oil. In certain embodiments, in addition to the apomorphine derivative, the formulation further contains a local anesthetic. The formulations described in the '503 patent can be modified as understood by one skilled in the art to contain other active drugs as described herein for use as described herein.

An injectable depot formulation is a dosage form, which is generally intended to have a therapeutic activity for 2 to 4 weeks after administration (e.g. neuroleptics like Fluphenazine decanoate in sesame oil). In order to maintain effective drug plasma levels the dosage form should release the drug at a more or less constant rate during the desired dosing interval. The difference between such prior art depots and depots used in the present invention is that the in accordance with the present invention, the drug is not needed to be absorbed into the systemic circulation.

A suitable form of depot preparation is the subcutaneous or intramuscular administration of an oil solution and/or oil suspension of a lipophilic drug. This gives a slow transport over the oil-biofluid interface and a slow dissolution in the biophase. Thus, when the drug is dissolved in a polar solvent (e.g. oils), which is non-miscible with the aqueous biological fluids, the drug has to be transported over the oil/water interface. When the oil/water partition coefficient is high, the transport will be slow. For very lipophilic drugs, the release from the oil phase may last for up to several weeks. The use of depot preparations such as those described herein may be used to deliver the drugs described herein for use as described herein.

The maximum volume of an oil solution/suspension to be injected intramuscularly or subcutaneously is 2-4 mL. This is feasible for the preparations of the aporphine derivatives of the present invention. The accumulated daily dose used in apomorphine s.c. therapy in Parkinson's disease is, e.g., 4-10 times about 1-4 mg (4-40 mg/day). For example, 2 mg Apomorphine HCl (or equivalent molar amount of another dopamine agonist(s), as the base or as a suitable salt or ion-pair) may be dissolved in 1 mL of an oil (sesame oil, Viscoleo or another approved oil) and the mixture gently heated (max 50° C.) shaken in a test tube shaker and ultrasonicated for a short time (minutes) until the mixture becomes a homogeneous solution or suspension. If necessary, the dopamine agonist may first be dissolved in 50-300 .mu.L DMSO, water, t-BuOH, PEG, benzylbenzoate, or another suitable and approved solvent or mixtures thereof, before adding the oil to a total volume of 1 mL.

Another example of a polymeric drug delivery system which may be adapted for use in the present invention by one skilled in the art is described in U.S. Pat. No. 5,601,835 (Sabel, et al.), hereby incorporated by reference, which describes a polymeric drug delivery system for delivery of any substance to the central nervous system. The delivery system is preferably implanted in the central nervous system for delivery of the drug directly to the central nervous system. These implantable devices can be used, for example, to achieve continuous delivery of dopamine, which cannot pass the blood brain barrier, directly into the brain for an extended time period. The implantable devices display controlled, “zero-order” release kinetics, a life time of a minimum of several weeks or months even when the devices contain water soluble, low molecular weight compounds, biocompatibility, and relative non-invasiveness. The polymeric devices are said to be applicable in the treatment of a variety of central nervous system disorders including Parkinson's disease, Alzheimer's dementia, Huntington's disease, epilepsy, trauma, stroke, depression and other types of neurological and psychiatric illnesses, and one skilled in the art can adapt that drug delivery system for delivering the drugs contemplated herein at the BONATH.

Yet another example of a system that may be adapted for use in the present invention is described in U.S. Pat. No. 5,601,835 (Sabel, et al.), hereby incorporated by reference, wherein a compound such as dopamine is encapsulated within a polymer to form a polymeric device, the device formed of a biocompatible polymer that is plastically deformable selected from the group consisting of ethylene vinyl acetate, polyurethanes, polystyrenes, polyamide, polyacrylamide, and combinations thereof having a non-porous polymer coating thereon with one or more openings, with limited water sorptivity and slight permeability to the passage of small, aqueous-soluble molecules, wherein said compound is linearly released (e.g., zero order release) from said polymeric device over a sustained period of time of at least 65 days at a predetermined level and rate when implanted in a patient at a specific site within the central nervous system where the compound is released directly into the central nervous system and the device remains essentially intact throughout the release period. The delivery device is a two-phase system which is manufactured using standard techniques such as blending, mixing or the equivalent thereof, following selection of the biologically active material to be delivered and an appropriate polymer for formation of the matrix. The general method of solvent casting as disclosed by Siegel and Langer, “Controlled release of polypeptides and other macromolecules”, Pharmaceutical Research 1, 2-10 (1984), is modified so that drug is dispersed within the devices to create channels and pores to the surface for release of the drug at the desired rate. Where appropriate, a coating impermeable to the drug is placed over a portion of the drug containing polymer matrix to further regulate the rate of release. One skilled in the art can adapt that drug delivery system for delivering the drugs contemplated herein and used as described herein.

Yet another formulation which may used to deliver the drug (e.g., dopamine agonists) as set forth in the present invention is described in U.S. Pat. No. 7,314,636 (Caseres, et al.), hereby incorporated by reference, which describes injectable implants comprising glycolic acid and bio-compatible/bio-absorbable polymeric particles containing a polymer of lactic acid. The particles are small enough to be injected through a needle but large enough to avoid engulfment by macrophages. The injectables of this invention may be in a pre-activated solid form or an activated form (e.g., injectable suspension or emulsion).

It is further contemplated that the system described in U.S. Pat. No. 6,586,006 (Roser, et al.), hereby incorporated by reference, can be adapted by one skilled in the art for use in the present invention for delivery of drugs as described herein. Therein are described delivery systems suitable for delivery of bioactive materials to subcutaneous and intradermal, intramuscular, intravenous tissue, the delivery system being sized and shaped for penetrating the epidermis. The delivery system comprises a vitreous vehicle loaded with the guest substance and capable of releasing the guest substance in situ at various controlled rates. Subdermal implantable therapeutic systems have also been formulated for slow release of certain pharmaceutical agents for extended periods of time such as months or years. A well-known example is Norplant® for delivery of steroid hormones.

In membrane permeation-type controlled drug delivery, the drug is encapsulated within a compartment that is enclosed by a rate-limiting polymeric membrane. The drug reservoir may contain either drug particles or a dispersion (or solution) of solid drug in a liquid or a matrix type dispersing medium. The polymeric membrane may be fabricated from a homogeneous or a heterogeneous nonporous polymeric material or a microporous or semipermeable membrane. The encapsulation of the drug reservoir inside the polymeric membrane may be accomplished by molding, encapsulation, microencapsulation, or other techniques. The implants release drugs by dissolution of the drug in the inner core and slow diffusion across the outer matrix. The drug release from this type of implantable therapeutic system should be relatively constant and is largely dependent on the dissolution rate of the drug in the polymeric membrane or the diffusion rate across or a microporous or semipermeable membrane. The inner core may substantially dissolve over time; however, in devices currently in use, the outer matrix does not dissolve.

Other implantable therapeutic systems involve matrix diffusion-type controlled drug delivery. The drug reservoir is formed by the homogeneous dispersion of drug particles throughout a lipophilic or hydrophilic polymer matrix. The dispersion of drug particles in the polymer matrix may be accomplished by blending the drug with a viscous liquid polymer or a semisolid polymer at room temperature, followed by cross-linking of the polymer, or by mixing the drug particles with a melted polymer at an elevated temperature. It can also be fabricated by dissolving the drug particles and/or the polymer in an organic solvent followed by mixing and evaporation of the solvent in a mold at an elevated temperature or under vacuum. The rate of drug release from this type of delivery device is not constant. Examples of this type of implantable therapeutic system are the contraceptive vaginal ring and Compudose implant. PCT/GB 90/00497 describes slow release glassy systems for formation of implantable devices. The described implants are bioabsorbable and need not be surgically removed. One skilled in the art can adapt these drug delivery systems for delivering the drugs contemplated herein.

In microreservoir dissolution-controlled drug delivery, the drug reservoir, which is a suspension of drug particles in an aqueous solution of a water-miscible polymer, forms a homogeneous dispersion of a multitude of discrete, unleachable, microscopic drug reservoirs in a polymer matrix. The microdispersion may be generated by using a high-energy-dispersing technique. Release of the drug from this type of drug delivery device follows either an interfacial partition or a matrix diffusion-controlled process. An example of this type of drug delivery device is the Syncro-Mate-C Implant.

Yet another formulation which may be adapted by one skilled in the art for use in the present invention is described in U.S. Pat. No. 6,576,263 (Truong, et al.), hereby incorporated by reference, which describes a preformed object for delivering an active agent for a subject, the preformed object including crosslinked protein, and methods of making and using.

Yet another formulation which may be adapted by one skilled in the art for use in the present invention is described in U.S. Pat. No. 6,287,588 (Shih, et al.), hereby incorporated by reference, which describes a composition and method for releasing a bio-active agent or a drug within a biological environment in a controlled manner. The composition is a dual phase polymeric agent-delivery composition comprising a continuous biocompatible gel phase, a discontinuous particulate phase comprising defined microparticles and an agent to be delivered. A microparticle containing a bio-active agent is releasably entrained within a biocompatible polymeric gel matrix. The bio-active agent release may be contained in the microparticle phase alone or in both the microparticles and the gel matrix. The release of the agent is prolonged over a period of time, and the delivery may be modulated and/or controlled. In addition, a second agent may be loaded in some of the microparticles and/or the gel matrix.

Yet another formulation which may be adapted by one skilled in the art for use in the present invention is described in U.S. Pat. No. 7,364,568 (Angel, et al.), hereby incorporated by reference, which describes a transdermal transport device includes a reservoir for holding a formulation of an active principle, and a needle with a bore extending along the length of the needle from a first end of the needle to a second end of the needle. The second end is substantially aligned to a plane parallel to a body surface of a biological body when the device is placed on the body surface. The device also includes an actuator which pumps the formulation through the bore of the needle between a target area of the body and the reservoir.

In yet other embodiments of the invention, the dopamine agonist is infused into the patient at the site of the injury using technology known to be useful for infusing other drugs, such as an insulin pump. One such system, U.S. Pat. No. 7,354,420 (Steil, et al.), hereby incorporated by reference, describes a closed loop infusion system controls the rate that fluid is infused into the body of a user. The closed loop infusion system includes a sensor system, a controller, and a delivery system. The sensor system includes a sensor for monitoring a condition of the user. The sensor produces a sensor signal, which is representative of the condition of the user. The sensor signal is used to generate a controller input. The controller uses the controller input to generate commands to operate the delivery system. The delivery system infuses a liquid into the user at a rate dictated by the commands from the controller. Preferably, the sensor system monitors the glucose concentration in the body of the user, and the liquid infused by the delivery system into the body of the user includes insulin.

The present invention is contemplated to encompass all implantable or injectable formulations, e.g., the technologies described above, with the inclusion of a drug(s) (e.g., dopamine agonist(s)), such that the administration of a drug useful for treatment of disease state or condition in humans via topical brainstem afferent stimulation (de-afferentation) therapy. Therefore, modifications of the invention via, e.g., the choice and/or amount of drug are considered to be obvious variations of this disclosure and within the scope of the appended claims.

### DETAILED DESCRIPTION OF PREFERRED EMBODIMENTS

The present invention will now be more fully described with reference to the accompanying examples. It should be understood, however, that the following description is illustrative only and should not be taken in any way as a restriction on the generality of the invention specified above.

### Example 1

### Topical Formulation

An aqueous based apomorphine cream was produced using Lipoderm® as the carrier. Lipoderm®/LIP is a commercially marketed compounding agent (from PCCA, Pharmaceutical Compounding Centers of America) having the following ingredients: Ethoxydiglycol, Water (Aqua), Glycerin, C12-15Alkyl Benzoate, Glyceryl Stearate, Dimethicone, Cetearyl Alcohol, Cetearyl Glucoside, Polyacrylamide, Cetyl Alcohol, Magnesium Aluminum Silicate, Xanthan Gum, Aloe Vera (Aloe Barbadensis), Tocopheryl Acetate (Vitamin E Acetate), Prunus Amygadalus Amara (Bitter Almond) Kernel Oil, Vitis Vinifera (Grape) Seed Extract, Triticum Vulgare (Wheat) Germ Oil, Retinyl Palmitate (Vitamin A Palmitate), Ascorbyl Palmitate (Vitamin C Palmitate), Pro-Lipo Multi-emulsion Liposomic System, Tetrasodium EDTA, Phenoxyethanol, and Sodium Hydroxymethylglycinate. Apomorphine is incorporated in an amount of 0.2%, and 4-AP in an amount of 0.5%.

### Example 2

### Topical Formulation

Into an aqueous based Lipoderm® as the carrier was incorporated the following drugs/active ingredients:

4-aminopyridine 0.5%

### Apomorphine 0.2%

Optional Active ingredients:

### Gabapentin 6%

### Prilocaine or Lidocaine 2.5%

Methylcobalamin (B12) 1000 mcg/gm;
methyl folate 800 mg/gm;
5-pyridoxal phosphate (B6) 0.5 gm/gm;
Benfotiamine (B1) 100 mg/gm;
R-alpha lipoic acid 100 mg/gm
Tramadol 1% (severe pain)

The formulation is utilized as follows. From about 2 to about 4 grams of the cream are applied to the affected area(s) from about 2 to about 4 times per day. In the case of diabetic neuropathy, the formulation may be applied, e.g., to the feet of the patient.

### Example 3

### Clinical Results

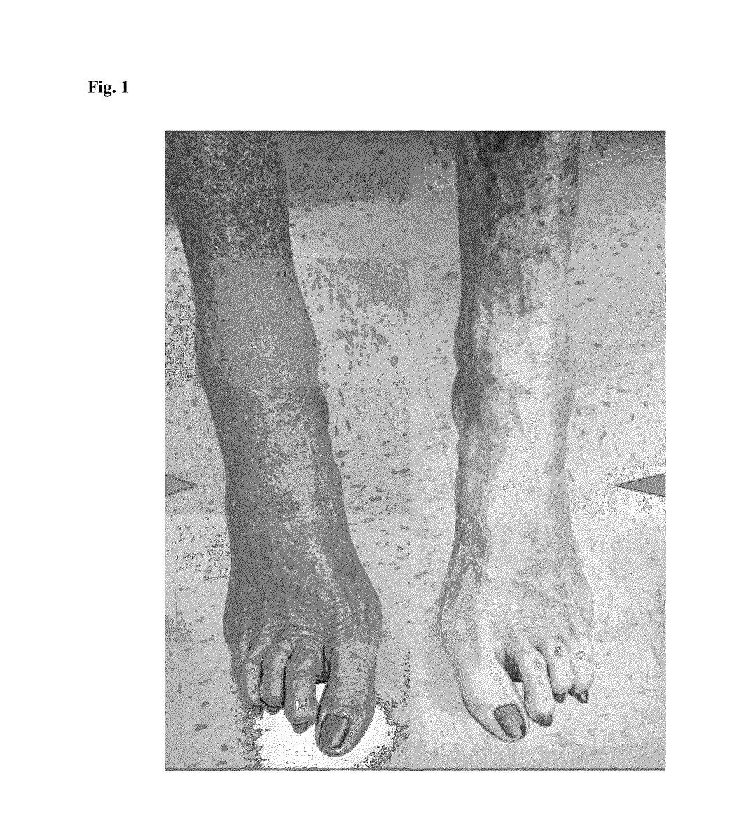

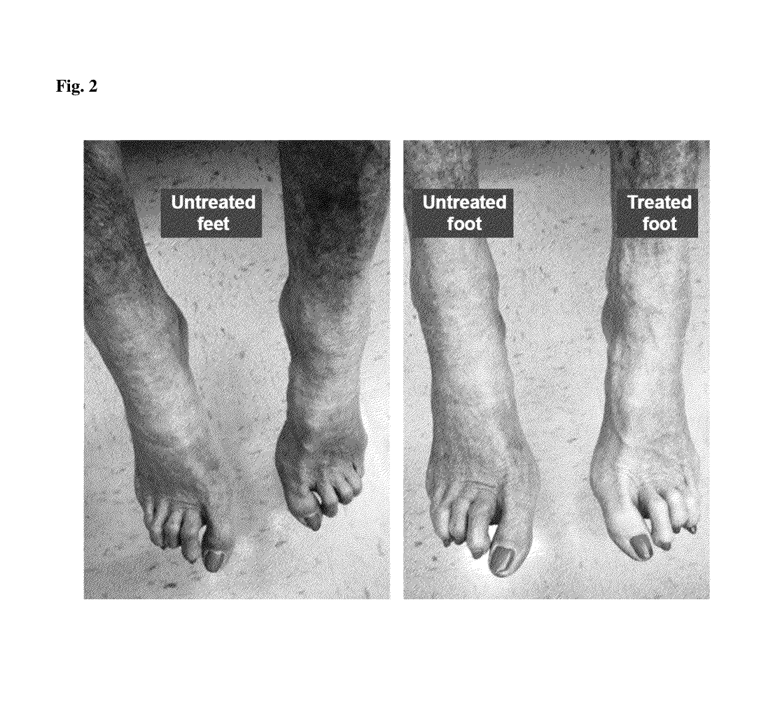

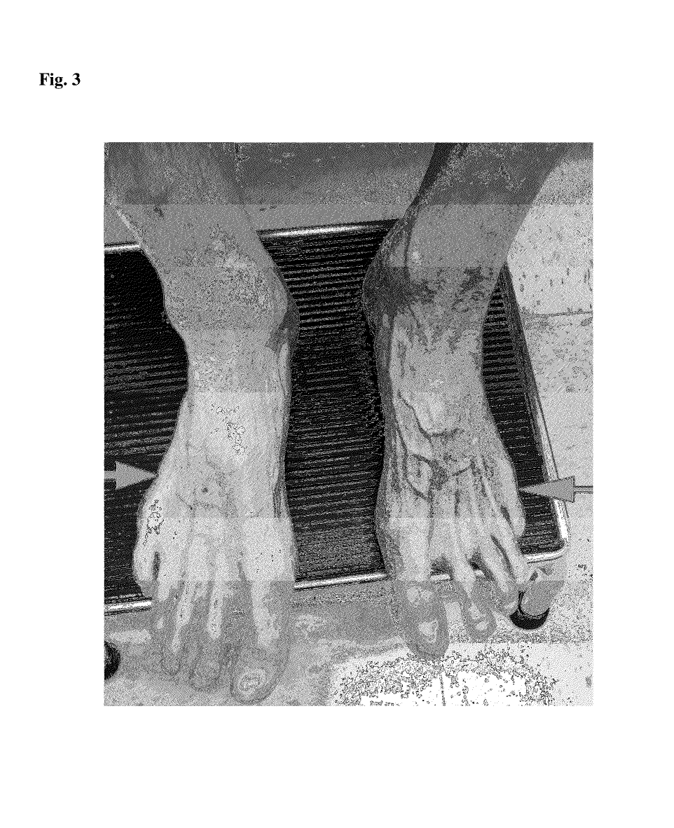

The efficacy of topical formulations were studied in human patients. For each of these patients, an amount of the compounded cream prepared in accordance with Example 1. The results are provided in Table B, and in FIGS. 1-3. FIG. 1 is a picture of the feet of a patient suffering from Charcot-Marie-Tooth peripheral neuropathy ten minutes after the patient was treated with topical 3 aminopyridine compounded cream to the left ankle and foot. FIG. 2 is a picture of the feet of a patient suffering from Charcot-Marie-Tooth peripheral neuropathy prior to and after the patient was treated with topical 4 amino pyridine compounded cream to the left ankle and foot. FIG. 3 is a picture of the feet of a patient suffering from diabetic peripheral neuropathy ten minutes after the patient was treated with topical 4 amino pyridine compounded cream to the right ankle and foot.

TABLE B
Treatment Of Peripheral Neuropathy With
Topical Drug And Neurotrophic* Combination Creams

| | | Duration of | | |
|---|---|---|---|---|
| Patient | Diagnosis | symptoms | Treatment | Results |
| R. H. | acute metabolic | Life-long with recent | 6 weeks Rx of topical | Pain, numbness and |
| 85 y/o male | neuropathy | worsening to extent could | 4-AP, apomorphine, | weakness improved. |
| | superimposed on | not ambulate without | gabapentin, lidocaine, | Ambulating without |
| | hereditary CMT | walker | prilocaine, and MFP* | walker or assistance |
| | neuropathy | | 3x/day. | |
| P. D. | Charcot-Marie-Tooth | 3 years of increasing pain, | 3 weeks of Rx of above | 20% improvement in |
| 87 y/o female | with recent increased | numbness, and weakness | combination 2x/day. | baseline pinprick and |
| | symptoms | with gait difficulties. | | temperature; no change |
| | | | | in vibratory sensation. |
| K. M. | diabetic/metabolic | 2 years of numbness, | 2 weeks of above | 80% relief of pain with |
| 64 y/o male | peripheral neuropathy | tingling, aching feet with | topical Rx nightly. | 20-30% |
| | with superimposed | restless legs and sleep | | improvement in |
| | lumbar radiculopathy | disturbance | | sensation within |
| | | | | 5-8 minutes of each |
| | | | | application. |
| | | | | Improved sleep. |
| C. A. | peripheral neuropathy | 4 years of bilateral foot | 1 week of above | Immediate significant |
| 70 y/o male | with entrapment | paresthesias with | topical | pain relief with |
| | neuropathy of feet | EMG/NCV documented | Rx continuing. | improved sensation in |
| | | peripheral neuropathy. Hx | | the feet. |
| | | of spinal and lower | | |
| | | extremity injuries from | | |
| | | severe auto accident with | | |
| | | coma from head injury. | | |
| C. M. | Diabetic peripheral | 3-4 years bilateral foot | 4-amino pyridine and | 50% reduction in |
| 75 y/o female | neuropathy | numbness, tingling, and | apomorphine topical | tingling with improved |
| | | pain: L > R. | cream. | sensation in 10-15 |
| | | | | minutes. |
| M. A. | familial peripheral | peripheral neuropathy | 2+ years (7/2011) of | With oral tramadol and |
| 90 y/o female | neuropathy and | diagnosed at | daily Rx with | topical neuropathy |
| | failed back syndrome | Mayo Clinic 1995 with | apomorphine and | cream, able to function |
| | | EMG/NCV. | tramadol cream 2x/d. | normally. |
| J. M. | peripheral neuropathy | symptoms of pain, | 4-AP, apomorphine, | peripheral neuropathic |
| 69 y/o female | with chemotherapy, | numbness, and weakness x | tramadol topical cream | symptoms stable with |
| | post-lumber | 2007, worse after chemo | with oral MFP | improvement of |
| | laminectomy. | for breast cancer 2009. | | numbness from |
| | | | | chemotherapy. |

*MFP neurotrophic support: methylcobalamin, methyl folate, and pyridoxal phosphate

### CONCLUSION

The examples provided above are not meant to be exclusive. Many other variations of the present invention would be obvious to those skilled in the art, and are contemplated to be within the scope of the appended claims.

The hypotheses of the inventor provided throughout the specification are for possible explanation purposes only, and are not meant to be limiting in any way.

1. A method of treating a peripheral neuropathic condition in humans, comprising topically administering a therapeutically effective amount of a drug selected from the group consisting of a dopamine agonist, a drug which enhances conduction of damaged demyelinated nerves, and a combination thereof at the site of the injury.

2. The method of claim 1, wherein the dopamine agonist is selected from the group consisting of apomorphine, pramipexole, ropinirole, bromocriptine, cabergoline, pergolide, rotigotine, entacapone, tocapone, seligiline, and mixtures of any of the foregoing.

3. The method of claim 1, wherein the drug which enhances conduction of damaged demyelinated nerves (e.g., a drug which blocks leaking potassium channels in damaged demyelinated nerves to improve conduction).

4. The method of claim 3, wherein the drug is 4-aminopyridine, 3,4 diaminopyridine, tetraethylammonium (TEA), or combinations of any of the foregoing.

5. The method of claim 3, wherein the drug which enhances conduction of damaged demyelinated nerves is 4-aminopyridine.

6. The method of claim 1, further comprising topically administering an additional drug(s) to the site of the injury, the additional drug selected from the group consisting of gabapentin, pregabalin, duloxetine, and combinations of any of the foregoing.

7. The method of claim 1, further comprising an additional agent(s) selected from the group consisting of L-methyl folate, methyl cobalamin, 5-pyridoxal phosphate, B1 benfotiamine, stabilized R-Alpha Lipoic Acid (R-ALA), gabapentin, pregabalin, pyridoxal 5′-phosphate, an opioid analgesic, a non-steroidal anti-inflammatory agent (NSAID), a local anesthetic, and mixtures of any of the foregoing.

8. The method of claim 1, wherein the drug is apomorphine.

9. The method of claim 1, further comprising an opioid agonist selected from the group consisting of alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, salts of any of the foregoing, and mixtures of any of the foregoing.

10. The method of claim 8, wherein the opioid agonist is tramadol in an amount from about 20 mg to about 40 mg.

11. The method of claim 8, wherein the opioid agonist is morphine sulfate in an amount from about 2.5 to about 5 mg.

12. The method of claim 1, wherein the drug is formulated in a pharmaceutically acceptable immediate release topical carrier.

13. The method of claim 11, wherein the carrier is aqueous-based.

14. The method of claim 1, further comprising applying a sufficient amount to the site of the injury such that the onset of clinical effect occurs in less than about 30 minutes.

15. The method of claim 1, wherein the carrier is a gel or cream.

16. The method of claim 14, further comprising adding at least one adjuvant selected from the group consisting of a penetration enhancer, anti-oxidant, stabilizer, and mixtures thereof to the carrier.

17-19. (canceled)

20. The method of claim 1, wherein the drug is administered via implantation or injection at the site of injury.

21-22. (canceled)

23. The method of claim 1, wherein the drug is incorporated into a delivery system selected from the group consisting of a gel, a matrix, microparticles, a pellet, an insert, a colloidal material, and mixtures of any of the foregoing.

24. The method of claim 1, wherein the drug is administered to create a depot under the skin at the site of the injury.

25. The method of claim 1, wherein a dopamine agonist is applied to the site of the injury, wherein the drug further comprises a skeletal muscle relaxant, an opioid agonist, an SNRI, a local anesthetic agent, an NMDA antagonist; and any combination thereof topically to the site of the injury.

26-39. (canceled)
